(19) United States
(12) Patent Application Publication
(10) Pub. No.: US 2007/0039077 A1
(43) Pub. Date: Feb. 15, 2007

(54) Endoscope

(75) Inventor: Satoshi Takami, Tokyo (JP)

Correspondence Address: GREENBLUM & BERNSTEIN, P.L.C., 1950 ROLAND CLARKE PLACE, RESTON, VA, 20191, US

(73) Assignee: PENTAX CORPORATION, Tokyo (JP)

(21) Appl. No.: 11/463,475

(22) Filed: Aug. 9, 2006

(30) Foreign Application Priority Data
Aug. 10, 2005 (JP) 2005-231711
Aug. 10, 2005 (JP) 2005-231730

(51) Int. Cl. A61B 1/00

(52) U.S. Cl. D24/138; 600/101

(57) ABSTRACT

An endoscope has a plurality of LEDs (Light-Emitting Diodes) and a light-controller. The plurality of LEDs illuminates an object, and is disposed at a tip portion of the endoscope. Further, in the plurality of LEDs, at least one first LED that emits white light and at least one second LED that emits light corresponding to a long-wavelength range in visible light, are included. The light-controller controls an emission of the plurality of LEDs.

### BACKGROUND OF THE INVENTION

1. Field of the Invention

The present invention relates to an endoscope that observes an object by using light irradiated from a light source. In particular, it relates to an endoscope that has LEDs (Light-Emitting Diodes) as a light source.

2. Description of the Related Art

In an endoscope with LEDs, light for illuminating an observed portion is irradiated from the LED provided at the distal end of a video-scope or fiber scope. An observed image is formed by the reflected light, and the operator diagnoses while seeing the observed image. For example, an LED emitting white-light is disposed at the distal end of the scope. Generally, the white LED is equipped with a blue LED emitting blue light and a fluorescent material. The white light is obtained by mixing the blue light and the fluorescent light, therefore, the white LED has spectral distribution characteristics that have a peak spectral value adjacent to a short-wavelength range in visible light.

### SUMMARY OF THE INVENTION

An object of the present invention is to provide an endoscope that is capable of clearly displaying an image of a reddish observed portion on a body-cavity.

An endoscope according to the present invention has a plurality of LEDs (Light-Emitting Diodes) and a light-controller. The plurality of LEDs illuminates an object, and is disposed at the tip portion of the endoscope. Further, in the plurality of LEDs, at least one first LED that emits white light and at least one second LED that emits light corresponding to a long-wavelength range in visible light, are included. The light-controller controls an emission of the plurality of LEDs. Since red light components are included in the irradiated light, the observed portion on the reddish body-cavity is clearly displayed.

### BRIEF DESCRIPTION OF THE DRAWINGS

The present invention will be better understood from the description of the preferred embodiments of the invention set forth below together with the accompanying drawings, in which:

FIG. 1 is a block diagram of a fiber scope according to a first embodiment;

FIG. 2 is a front view of the tip portion of the fiber-scope;

FIG. 3 is a block diagram of a light-controller;

FIG. 4 is a view showing spectral distribution characteristics of LEDs;

FIG. 5 is a view showing spectral transmitting characteristics of a plastic image-fiber;

FIG. 6 is a view showing an electronic endoscope according to a second embodiment;

FIG. 7 is a block diagram of a fiber-scope according to a third embodiment;

FIG. 8 is a front view of the tip surface of the fiber scope;

FIG. 9 is a schematic side view of the tip surface of the fiber-scope;

FIG. 10 is a block diagram of the light-controller according to the third embodiment; and

FIG. 11 is a block diagram of an electronic endoscope according to a fourth embodiment.

### DESCRIPTION OF THE PREFERRED EMBODIMENTS

Hereinafter, the preferred embodiments of the present invention are described with reference to the attached drawings.

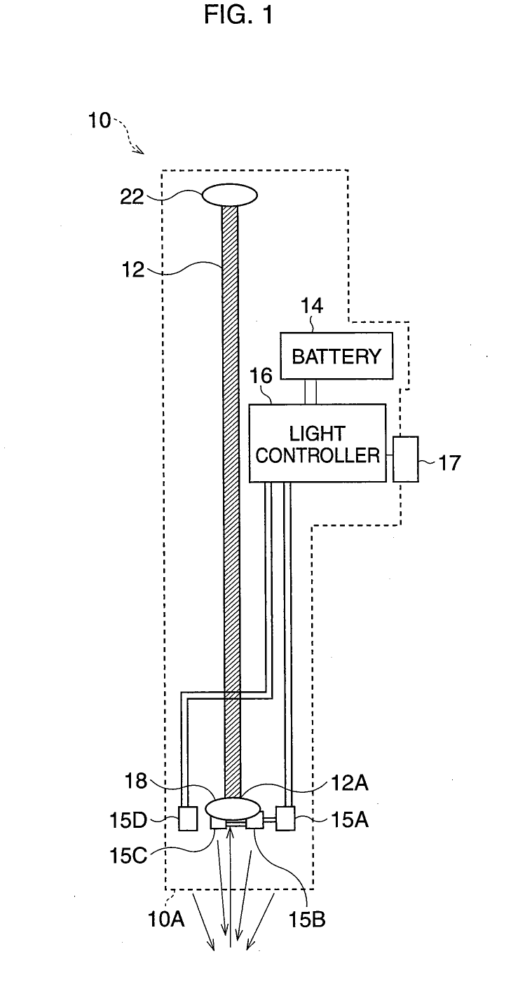

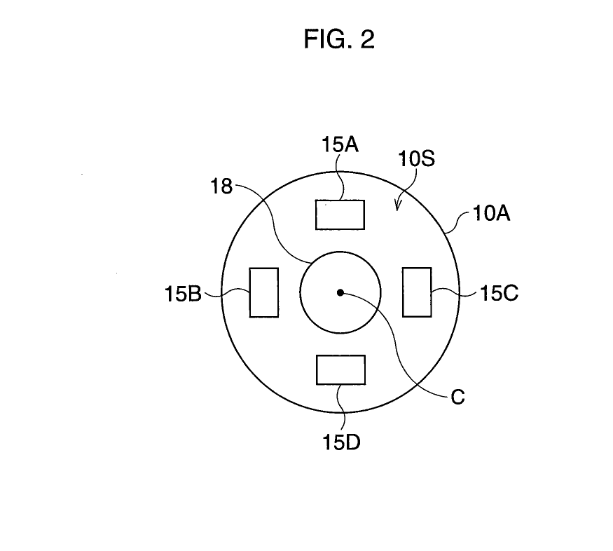

FIG. 1 is a block diagram of a fiber scope according to a first embodiment. FIG. 2 is a front view of the tip portion of the fiber scope.

A fiber scope 10 has an image-fiber 12 of a fiber-optic bundle, and has a plurality of LEDs 15A to 15D, which are disposed at the tip portion of the fiber scope 10. Each of the LEDs 15A to 15D is covered with resin-lens. A battery 14 supplies electrical power to a light-controller 16. The light-controller 16 turns the LEDs 15A to 15D ON in accordance with the electric power supply.

As shown in FIG. 2, the LEDs 15A to 15D are arranged so as to be symmetrical with respect to the center axis C of the tip portion 10A, and are arranged around an objective lens 18 at regular intervals. Each of the LEDs 15A, 15B, and 15C is a white LED that emits white light toward the direction that the tip surface 10S of the tip portion 10A faces. On the other hand, the LED 15D is a red LED that emits red light toward the direction that the tip surface 10S faces. When the LEDs 15A to 15D are turned ON, light emitted from the LEDs 15A to 15D passes through a diffusion lens (not shown), and is irradiated from the tip portion 10A. Consequently, an observed portion is illuminated by the irradiated light.

Light reflected off the observed portion passes through the objective lens 18 so that an observed image is formed on the tip surface 12A of the image-fiber 12 shown in FIG. 1. The plastic image-fiber 12 optically transmits the observed image to the opposite tip surface of the image-fiber 12. The operator watches the observed image via an eyepiece 22. A color balance button 17 is a button for changing a resistor value of a variable resistor (herein, not shown). The operator sets the resistor value by manipulating the color balance button 17.

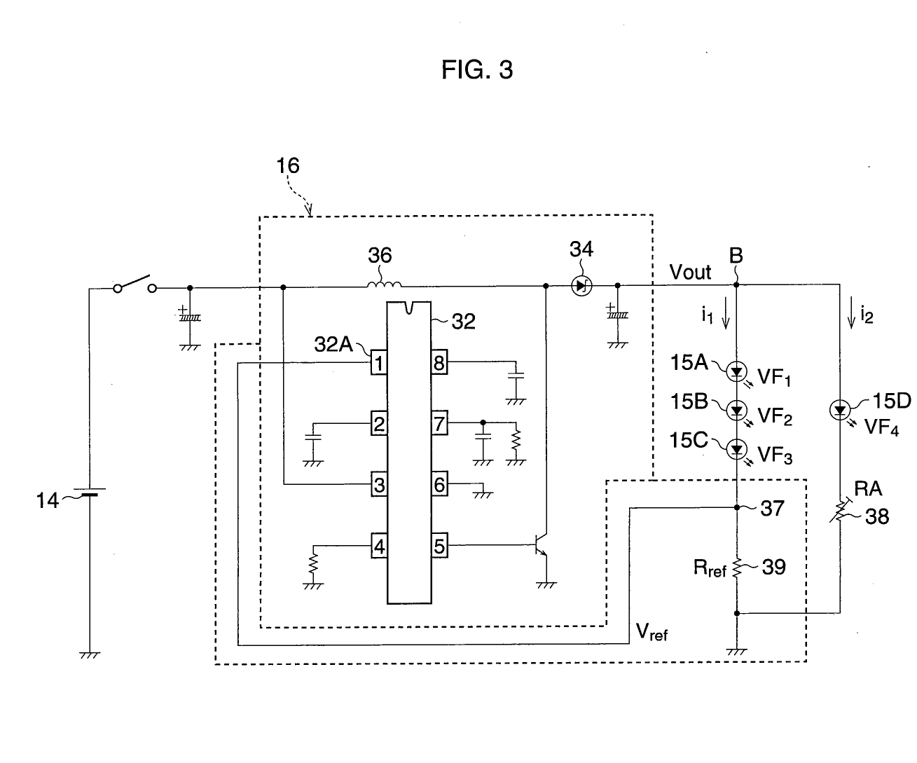

FIG. 3 is a block diagram of the light-controller 16. The light-controller 16 has an electric power controller 32, which functions as a DC/DC converter. An input voltage from the battery 14 is step-upped by the electric power controller 32, and increased voltage is output to the LEDs 15A to 15D via an inductor 36 and a diode 34. The electric power controller 32 stabilizes an output voltage Vₒᵤₜ while monitoring the voltage at a connecting point 37; namely, a terminal 32A of the controller 32.

The LED 15D connects with the LEDs 15A to 15C in parallel with respect to the light-controller 16. Electric currents i₁ and i₂ flow through the LEDs 15A to 15C and the LED 15D, respectively. A resistor 39 having the resistor value R_{ref} connects with the LEDs 15A to 15C in a series, and a variable resistor 38 having a variable resistor value RA connects with the LED 15D. The electric circuit branches from the connecting point 37 to the electric power controller 32.

When a standard voltage for controlling an output voltage Vₒᵤₜ is designated by “V_{ref}”, the voltages across the LEDs 15A to 15C are designated by “VF1”, “VF2”, and “VF3”, respectively; the voltage across the LED 15D is designated by “VF4”, and the voltage across the variable resistor 38 is designated by “VRA”, the following equation is satisfied:
Vₒᵤₜ=VRA+VF4=V_{ref}+VF1+VF2+VF3 (1)
Therefore, the electric currents i₁ and i₂ satisfy the following equation:
Vₒᵤₜ=i₂×RA+VF4=i₁×R_{ref}+VF1+VF2+VF3 (2)
Since the values of the voltages VF1, VF2, VF3, and VF4 are substantially equal to one another, the difference between the electric current i₁ and the electric current i₂ occurs by changing the resistor value RA of the variable resistor 38. Therefore, by adjusting the resistor value RA, the light-intensity of the LED 15D increases so that light, in which reddish light is relatively stronger than white light, illuminates the observed portion. The resistor value RA is set by operating the color balance button 17, shown in FIG. 1.

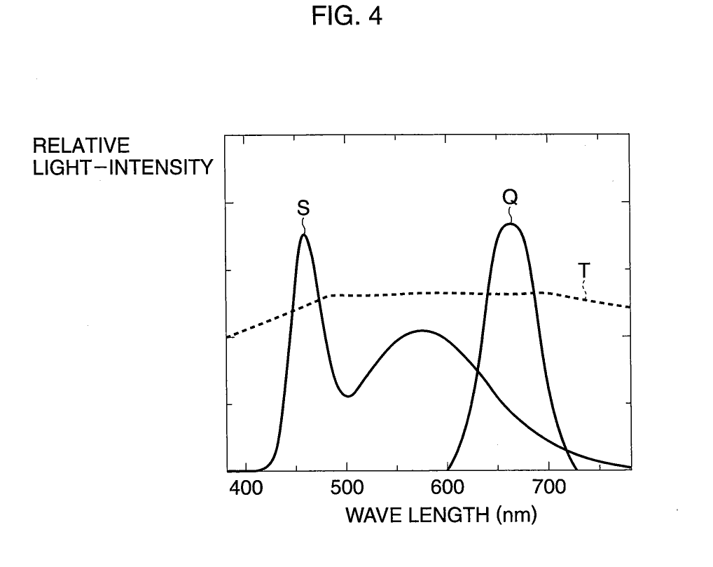

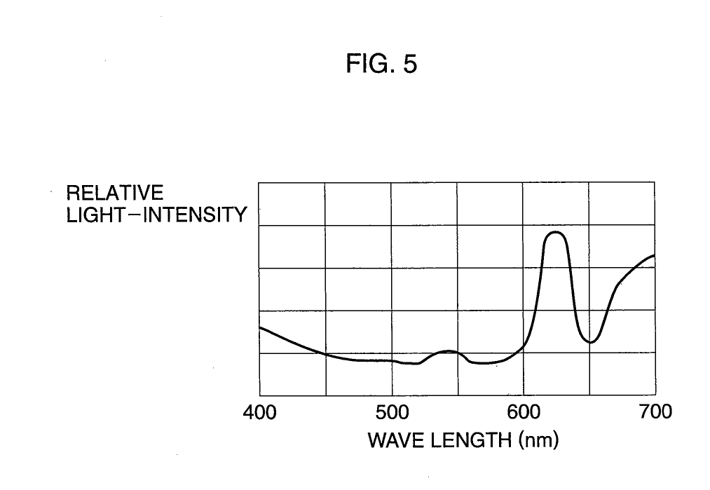

FIG. 4 is a view showing the spectral distribution characteristics of the LEDs 15A to 15C. FIG. 5 is a view showing the spectral transmitting characteristics of the plastic image-fiber 12.

As shown in FIG. 4, the spectral distribution characteristics of the LEDs 15A to 15C are represented by the spectral curved line S, which is distributed over the wavelength of visible light, and has a peak level in a short-wavelength range that includes blue light (480 nm). On the spectral distribution curved line “S”, spectral values in the long-wavelength range including red light are relatively small compared to those in the short-wavelength range. This spectral curved line is different from a spectral line of a general light source that emits white light, which is shown by the broken line T. On the other hand, the spectral distribution characteristics of the LED 15D, which are represented by the spectral curved line Q, have a peak level in the long-wavelength range. Therefore, light obtained by the mixture of the four LEDs 15A to 15D includes more reddish light spectral components than other light spectral components.

As shown in FIG. 5, the plastic image fiber 12 has the spectral transmitting characteristics that transmit more light, which corresponds to the long-wavelength range, than light corresponding to the short-wavelength range. Therefore, the red light components in the reflected light pass through the image fiber 12 without loss.

In this way, in the first embodiment, the white LEDs 15A to 15C and the red LED 15D are provided at the tip portion 10A of the endoscope 10. The observed portion on the body-cavity is reddish and has the spectral reflecting characteristics that reflect light corresponding to the long-wavelength range more than light corresponding to the short-wavelength range. Since the light irradiated from the LEDs 15A to 15D includes a high proportion of red light components, a reddish observed portion is clearly formed on the incidence surface of the image-fiber. Then, the reflected light is transmitted by the plastic image-fiber 12 without loss. Further, in accordance with a target for diagnosis, or operating condition, the ratio of the red light components can be changed by operating the color balance button 17.

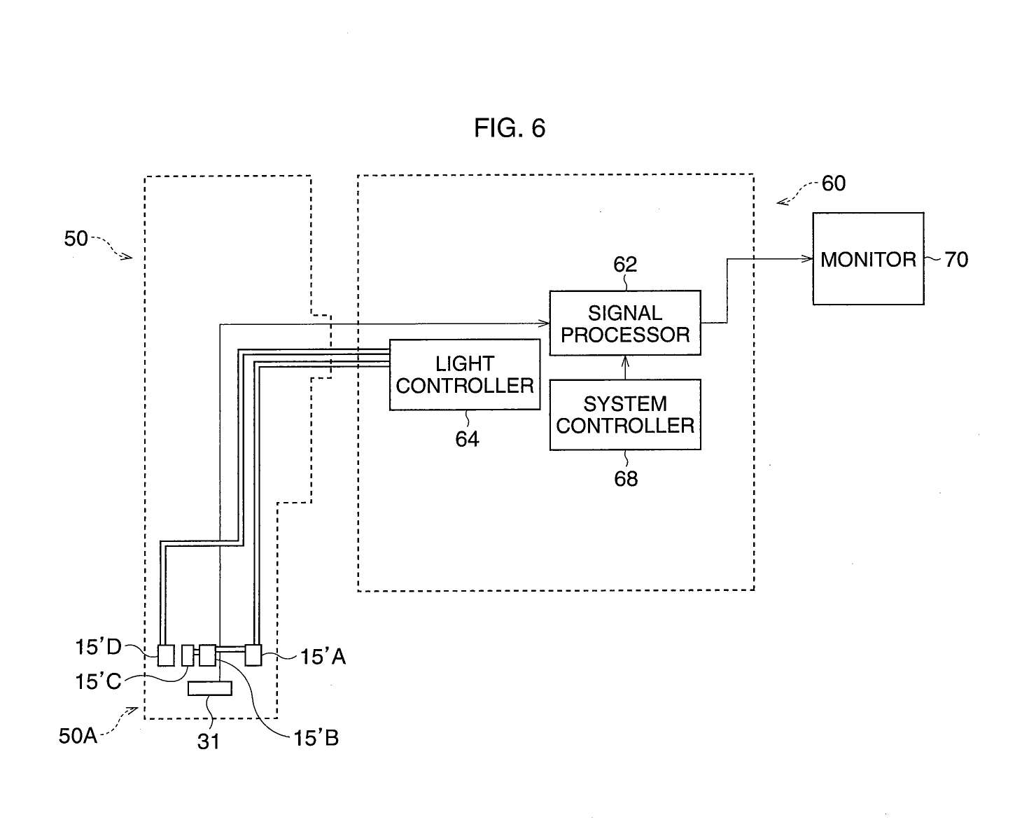

FIG. 6 is a view showing an electronic endoscope according to a second embodiment. The electronic endoscope has a video-scope 50 and a video-processor 60, and a monitor 70 is connected to the video-processor. Four LEDs 15′A, 15′B, 15′C, and 15′D are provided at the tip portion 50A of the video-scope 50, and are controlled by a light-controller 64 in the video-processor 60. A system control circuit 68 controls a signal processor 62 and the light-controller 64. Each of the LEDs 15′A, 15′B, and 15′C emits white light, whereas the LED 15′D emits red light. An object image is formed on a CCD 31, and image-pixel signals are fed from the CCD 31 to the signal processor 62. In the signal processor 62, video signals are generated and are fed to the monitor 70; thus, the object image is displayed on the monitor 70.

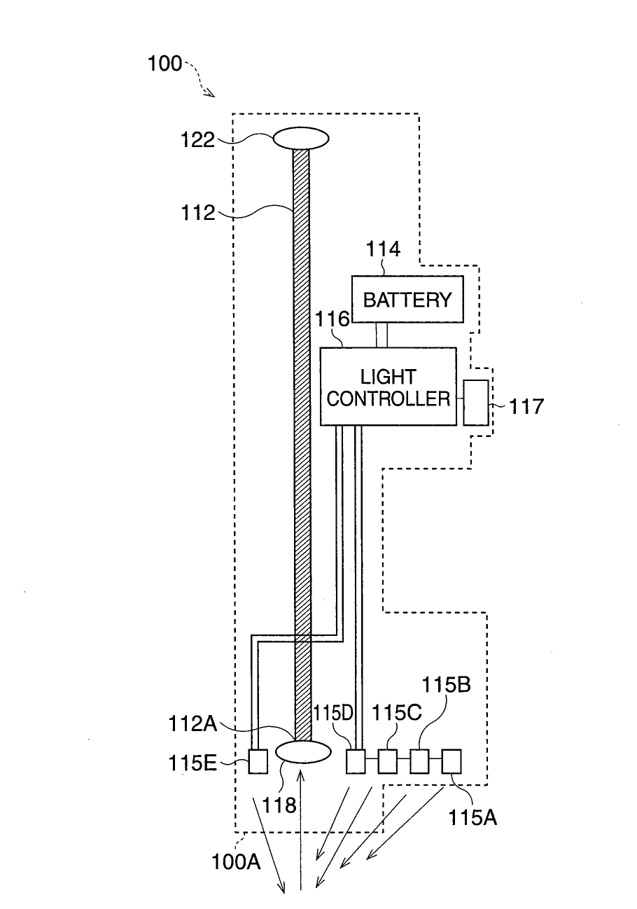

With reference to FIGS. 7 to 9, a third embodiment is explained. The third embodiment is different from the first embodiment in that a red LED emits red light toward a white LED.

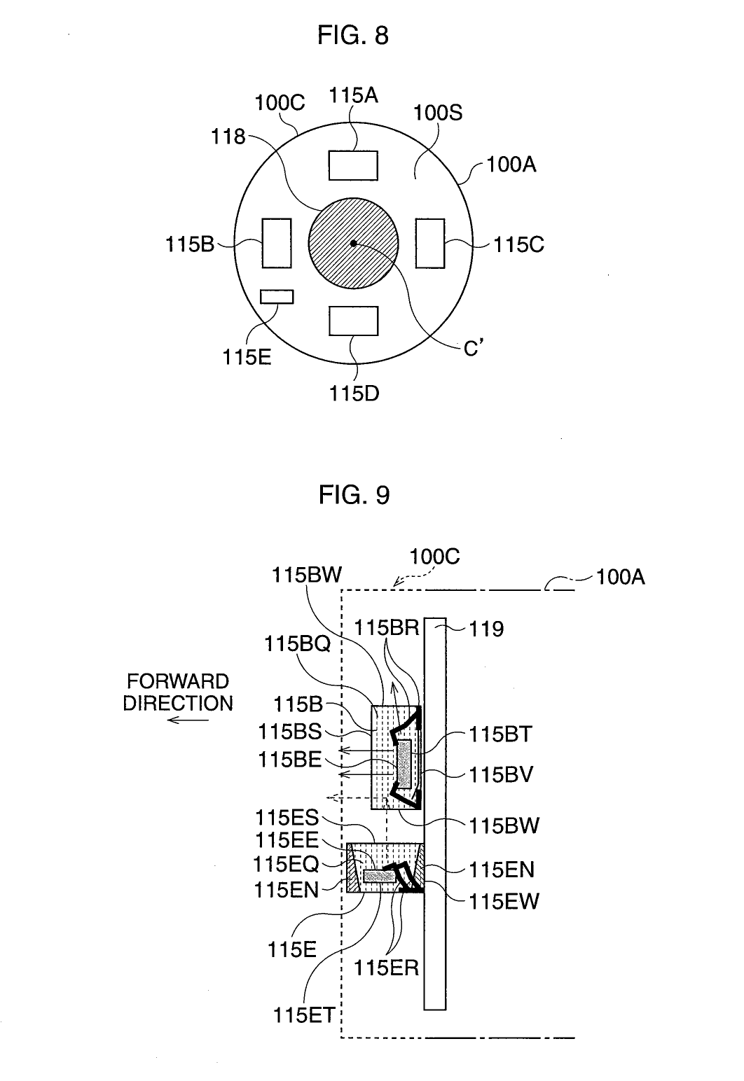

FIG. 7 is a block diagram of a fiber scope according to the third embodiment. FIG. 8 is a front view of the tip surface of the fiber scope. FIG. 9 is a schematic side view of the tip surface of the fiber scope.

The fiber scope 100 has an image-fiber 112, a battery 114, a light-controller 116, and five plate-like LEDs 115A to 115E. The four LEDs 115A to 115D are white LEDs that emit white light, whereas the remaining LED 115E is a red LED that emits red light. As shown in FIG. 8, the four LEDs 115A to 115D are disposed so as to have symmetry with respect to the center axis C′ of the tip portion 100A of the fiber scope 100, and are arranged around an objective lens 118 at regular intervals. The LED 115E is located adjacent to the LED 115B so as to face the LED 115B. A transparent cover 100C is attached to the tip portion 110A of the fiber scope 100, and an outer surface of the cover 100C is formed as the tip surface 100S of the tip portion 100A.

Light irradiated from the LEDs 115A to 115E is reflected off an observed portion, and the reflected light passes through the objective lens 118, so that an object image is formed on the tip surface 112A of the image-fiber 112 shown in FIG. 7. The image-fiber 112 transmits the object image optically, and the operator watches the object image via an eyepiece 122. A color balance button 117 is operated when changing a resistor value of a variable resistor (herein not shown).

In FIG. 9, the LED 115B and the LED 115E are illustrated (the LEDs 115A, 115C, and 115D are omitted). The LED 115B has a diode element 115BT and a light-diffusion resin 115BQ, which is loaded in the LED 115B so as to encompasses the diode device 115BT. The resin 115BQ is herein epoxy resin. Similarly, the LED 115E has a diode element 115ET and a light-diffusion resin 115EQ, which is loaded in the LED 115E so as to encompasses the diode element 115ET.

The LED 115B has an irradiation surface 115BS and a bottom surface 115BV, which contacts with a substrate 119 such that the diode element 115BT is opposite the substrate 119. A lead frame 115BR is connected to an outer surface 115BE of the diode element 115BT to emit the white light from the irradiation surface 115BS toward the forward direction of the tip portion 110A. Further, the light irradiated from the diode element 115BT is diffused by the light-diffusion resin 115BQ so that the irradiated light is emitted toward a surrounding direction via a side surface 115BW. The LEDs 115A, 115C, and 115D are constructed similarly to the LED 115B.

On the other hand, the red LED 115E is attached to the substrate 119 such that the side surface 115EN of the LED 115E contacts with the substrate 119, and an irradiation surface 115ES of the LED 115E faces the side surface 115BW of the LED 115B. A lead frame 115ER is connected to an outer surface 115EE of the diode element 115ET. Thus, the red light emitted from the diode element 115ET exits from the irradiation surface 115ES, and enters into the LED 115B via the side surface 115BW. The entered red light is diffused by the light-diffusion resin 115BQ so that the red light is irradiated from the irradiation surface 115BS and the side surface 115BW of the LED 115B. A mirror 115EN is arranged at both sides of the diode element 115ET so that the directivity of the emitted red light becomes broad.

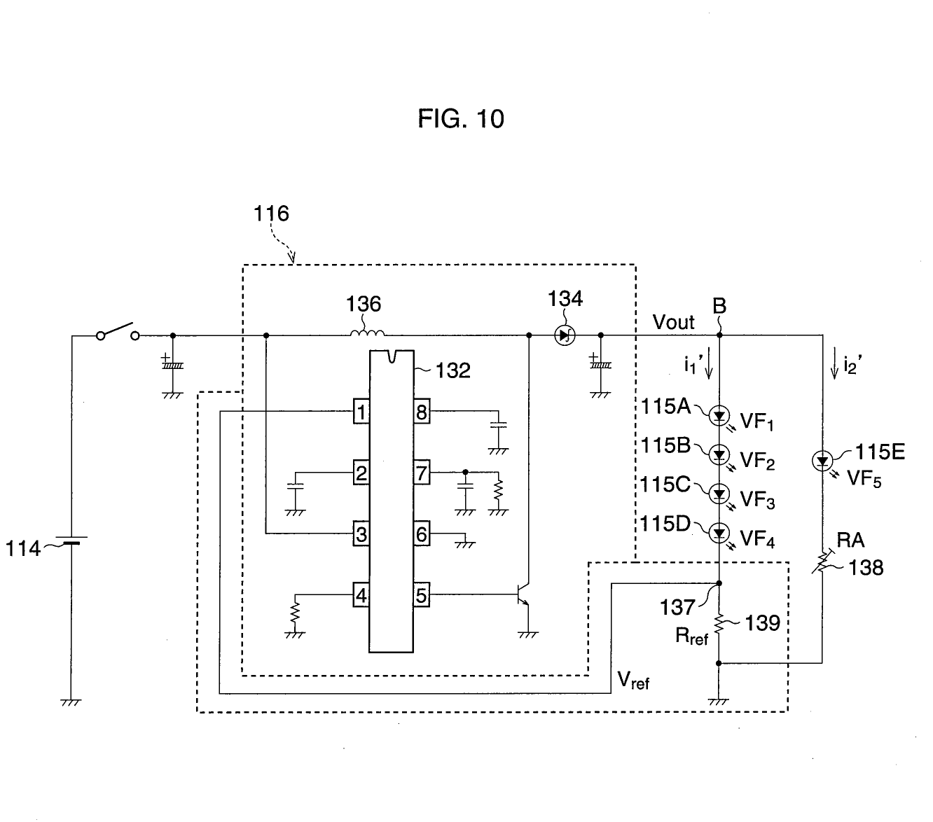

FIG. 10 is a block diagram of the light-controller 116 according to the third embodiment.

The light-controller 116 has an electric power controller 132, an inductor 136, and a diode 134. Input voltage from the battery 114 is output to the LEDs 115A to 115D via the inductor 136 and the diode 134. A resistor 139 having a resistor value R_{ref} connects with the LEDs 115A to 115D in a series, whereas a variable resistor 138 having a resistor value RA connects with the LED 115E.

When a standard voltage for controlling an output voltage Vₒᵤₜ is designated by “V_{ref}”; the forward voltages of the LEDs 115A to 115D are designated by “VF1”, “VF2”, “VF3”, and “VF4”, respectively; the voltage of the LED 115E is designated by “VF5”, and the voltage across the variable resistor 138 is designated by “VRA”; the following equation is satisfied:
Vₒᵤₜ=VRA+VF5=V_{ref}+VF1+VF2+VF3+VF4 (3)
Therefore, the electric currents i₁, and i₂, satisfy the following equation:

$${V}_{out} = {i}_{{2}^{′}} \times RA + VF 5 = {i}_{{1}^{′}} \times {R}_{ref} + VF 1 + VF 2 + VF 3 + VF 4 \qquad ( 4 )$$

Therefore, similarly to the first embodiment, by adjusting the resistor value RA of the variable resistor 138, the light-intensity of the LED 115E is changed.

In this manner, in the third embodiment, the four white LEDs 115A to 115D are disposed at the tip portion 100A of the endoscope 100 so as to emit the white light toward the forward direction, and the red LED 115E is arranged adjacent to the LED 115B so as to be opposite the side surface 115BW of the LED 115B. Thus, the red light enters into the LED 115B, and the light emitted from the LEDs 115A to 115D and the light emitted from the LED 115E are mixed.

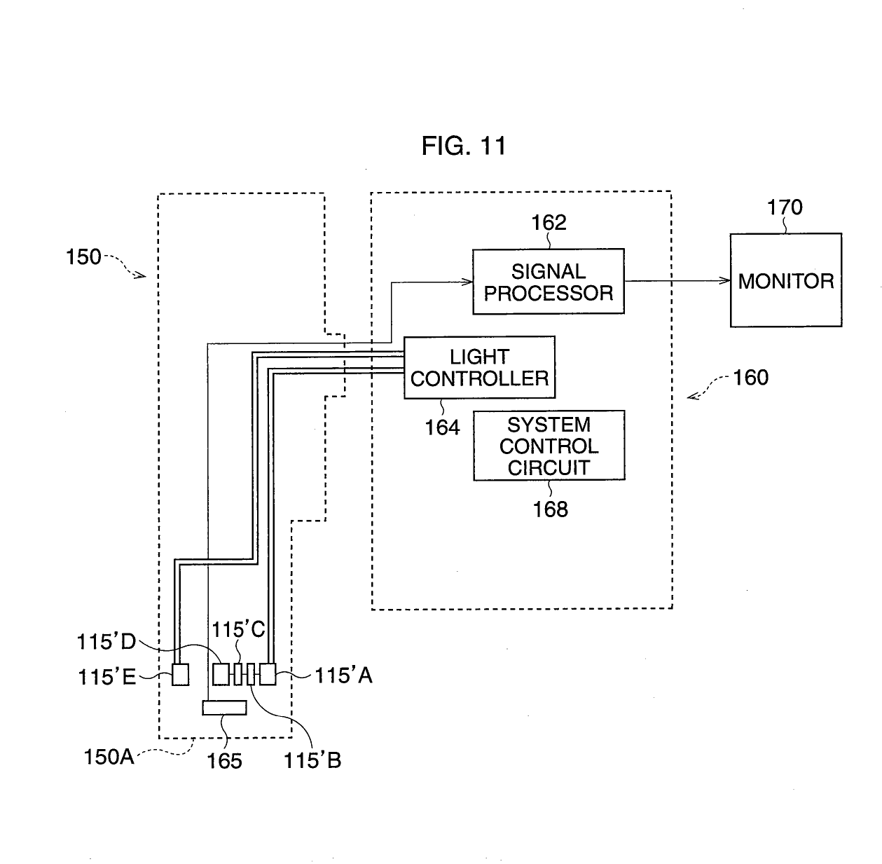

FIG. 11 is a block diagram of an electronic endoscope according to a fourth embodiment. The electronic endoscope is equipped with a video-scope 150 and a video-processor 160. A monitor 170 is connected to the video-processor 160. The video-processor 160 has a signal processor 162, a light-controller 164, and a system control circuit 168. The video-scope 150 has five LEDs 115′A to 115′E, and a CCD 165.

Optionally, an image fiber composed of a glass fiber can be used instead of the plastic image-fiber 12. Optionally, shell-shaped LEDs, or chip-shaped LED may be used instead of the plate-like LEDs. An LED that emits light corresponding to the long-wavelength range in visible light may be used, instead of the red LED. Optionally, the LEDs may be provided in the processor. In this case, a light-guide composed of a fiber-optic bundle is used.

Finally, it will be understood by those skilled in the arts that the foregoing description is of preferred embodiments of the device, and that various changes and modifications may be made to the present invention without departing from the spirit and scope thereof.

The present disclosure relates to subject matter contained in Japanese Patent Applications No. 2005-231711 and No. 2005-231730 (both filed on Aug. 10, 2005), which are expressly incorporated herein, by reference, in their entireties.

1. An endoscope comprising:
a plurality of LEDs (Light-Emitting Diodes) that illuminates an object and is disposed at a tip portion of said endoscope, said plurality of LEDs comprising at least one first LED that emits white light and at least one second LED that emits light corresponding to a long-wavelength range in visible light; and
a light-controller that controls an emission of said plurality of LEDs.

2. The endoscope of claim 1, further comprising an image-fiber that transmits an object image optically.

3. The endoscope of claim 2, wherein said image-fiber is a plastic optical fiber that has spectral transmitting characteristics that transmit the light corresponding to a long-wavelength range in visible light more than other light.

4. The endoscope of claim 1, further comprising an video-scope with an image sensor.

5. The endoscope of claim 1, wherein said first LED comprises a blue LED that emits blue light and a fluorescent material.

6. The endoscope of claim 1, wherein said second LED comprises a red LED that emits red light.

7. The endoscope of claim 1, further comprising a variable resistor that connects with said light-controller in a series, said first LED connecting with said second LED in parallel with respect to said light-controller.

8. The endoscope of claim 7, further comprising a resistor value setter that sets a value of said variable resistor.

9. The endoscope of claim 1, wherein said first LED comprises a resin that diffuses light passing through said resin, said first LED emitting the white light toward a forward direction of the tip portion, said second LED emitting the light corresponding to a long-wavelength range in visible light toward said first LED.

10. The endoscope of claim 9, wherein said first LED comprises a first diode element that emits the white light toward the forward direction, said resin covering said first diode element.

11. The endoscope of claim 9, wherein said first LED and said second LED are arranged along the tip surface of the tip portion.

12. The endoscope of claim 11, wherein said second LED emits the light corresponding to a long-wavelength range in visible light toward a side of said first LED.

13. The endoscope of claim 12, wherein said second LED comprises a second diode element that emits the specific light and is located so as to emit the light corresponding to a long-wavelength range in visible light along the tip surface of the tip portion.

14. The endoscope of claim 11, wherein said second LED comprises an irradiation surface that faces a direction along the tip surface of the tip portion.

15. The endoscope of claim 9, wherein said second LED is located adjacent to said first LED.

16. The endoscope of claim 9, wherein a plurality of first LEDs are regularly arranged at given intervals.

17. An apparatus for illuminating an observed portion on a body-cavity, comprising:
at least one first LED that emits white light; and
at least one second LED that emits light corresponding to a long-wavelength range in visible light.
